(19) United States
(12) Patent Application Publication
(10) Pub. No.: US 2011/0092935 A1
(43) Pub. Date: Apr. 21, 2011

(54) FABRIC LINER FOR SKIN-CONTACTING ITEMS

(75) Inventor: Elly Hann, La Jolla, CA (US)

(73) Assignee: PERSIMMON SCIENTIFIC, INC., La Jolla, CA (US)

(21) Appl. No.: 12/973,407

(22) Filed: Dec. 20, 2010

Related U.S. Application Data
(63) Continuation-in-part of application No. 12/618,584, filed on Nov. 13, 2009.
(60) Provisional application No. 61/114,067, filed on Nov. 13, 2008.

(51) Int. Cl. A61F 13/53; A61L 15/46

(52) U.S. Cl. 604/367; 604/360; 604/372

(57) ABSTRACT

A liner for use in skin-contacting garments and other objects that contact the skin has a moisture-wicking layer formed from a non-shearing, moisture-vapor permeable woven fabric of synthetic filament yarns which is treated with an anti-microbial substance. Additional layers may include a breathable, highly-absorbent microfiber fabric that draws moisture from the inner surface to the outer surface of the fabric where it may be retained, and a moisture transport layer. If multiple layers are used, all layers may be stitched together with a seam around the outer edges of the layers. The liner may be shaped to fit within a garment or other wearable-article, such as a bra cup, diapers and underpants.

### RELATED APPLICATIONS

This application is a continuation-in-part of application Ser. No. 12/618,584, filed Nov. 13, 2009, which claims the priority of U.S. Provisional Application No. 61/114,067, filed on Nov. 13, 2008, which applications are incorporated herein by reference.

### BACKGROUND OF THE INVENTION

Many situations arise in which a person's skin must remain in contact with a textile for extended periods of time (from minutes to hours) after the contact area has become wet or moist due to release of a liquid that is produced by a bodily function. Such functions include perspiration, urination, lactation, and discharge from wounds or certain skin conditions. While the mechanisms and fluids are different, a common concern with each is that continued contact between the wet fabric and the skin can lead to skin irritation, abrasion and possible infection.

In the example of diapers, whether for children who are not yet toilet-trained or for incontinent adults, one of the most common approaches to increasing the comfort of the wearer is to provide a disposable garment of layered construction that allows the transfer and distribution of urine to an absorbent core structure where it is locked in. Basic layers are an outer shell of polyethylene film or a non-woven and film composite which prevents wetness and soil transfer, an inner absorbent layer of a mixture of air-laid paper and superabsorbent polymers for wetness, and a layer nearest the skin of non-woven material with a distribution layer directly beneath which transfers wetness to the absorbent layer. While efforts have been made to make the skin contact layer with a smooth surface by using a melt blowing process, it is still made of a plastic (typically polypropylene) and may not be the ideal material for comfort and smoothness against the skin, especially is the skin is sensitive and/or already irritated.

Another example of a skin-contacting fabric product is nursing pads. While not every nursing mother experiences leaking of breast milk, many do, requiring the use of a nursing bra pad in conjunction with a nursing bra. Nursing bra pads, which are placed against the inside surface of each bra cup, are available in both washable and disposable forms. Disposable pads are typically made of layers of non-woven paper or synthetics with a super-absorbent diaper-like material in the middle layer and often include a plastic lining to prevent moisture from being absorbed into the bra cup. Unless the plastic liner is breathable, it can impede the healing of traumatized nipples or make nipple infections or breast yeast infections more likely. Since the pads should be changed when they become wet, they may need to be changed frequently, which could become expensive. Furthermore, women who are environmentally conscious may find disposal of large numbers of plastic lined, slowly biodegradable pads to be objectionable.

Washable pads, which are typically made of 100% cotton, such as flannel, or hemp, tend to be less expensive in the long run than disposable, however a ready supply of clean, dry pads must be kept on hand for changing. Care must be taken during washing to avoid reduction of the pad's absorbency, and fabric softener is not recommended for this reason. On the other hand, repeated washings without fabric softener could cause the fabric to lose its softness, increasing shear and friction, and reducing comfort. Available washable pads suffer from abrasive surface, lack of breathability, and/or poor ability to conform to the breast shape. Odor control and moisture wicking can be problems as well.

Other commercially available nursing pads include 100% wool pads, which tend to be bulky and relatively expensive, and must be hand washed.

Yet another example of fabrics that contact sensitive skin areas is in the area of prostheses. A common prosthetic is the type used by women who have undergone mastectomies. For such women, the ability to use a prosthetic is important to their psychological healing after a drastic surgery that can frequently impact a woman's self-image. These women may suffer from chest wall irritation due to ill-fitting mastectomy bras, poor air circulation between the chest wall and the prosthesis, moisture build-up, chafing and fungal infections. Many mastectomy bras include a pocket into which the prosthetic can be inserted. A disadvantage to this approach is that the woman has a limited selection of bra styles, and she may be prevented from choosing a bra that is more attractive.

Other situations in which fabrics that are in contact with the skin may become moistened with bodily fluids and/or other externally-based moisture include athletic garments and equipment, medical dressings such as bandages, liners and sleeves for prosthetic limbs, and many others. Improved comfort, decreased risk of irritation and infection, and odor control are desirable characteristics for many such products. The present invention is directed to such improvements.

### SUMMARY OF THE INVENTION

The present invention provides alternatives to current fabrics that are used to create skin-contacting garments, liners, covers and pads. In an exemplary embodiment, the inventive liner is a multi-layered textile assembly that possesses all of the qualities that are important for providing improved comfort, moisture handling, breathability, shear-reducing, rapid-drying and anti-microbial, while being durable, launderable and lightweight.

In a first application of the inventive liner, a diaper liner is formed from an elongated strip, generally rounded at the ends to form an oval shape or a semi-hourglass shape, i.e., contoured to fit smoothly along the crotch portion of the diaper or underpants. The composite textile has a first, skin contact layer formed from a woven fabric of synthetic filament yarns woven to provide air and moisture vapor permeability and a non-shearing surface, with a thickness of less than 1 mm, the skin contact layer having an inner, skin contact surface and a bottom surface; a second layer formed from a breathable microfiber fabric woven from synthetic fibers having a cross-sectional shape that includes a plurality of voids for generating a capillary effect for moisture transfer and conduction from one surface to the opposite, outer surface of the fabric. Since the outer layer of the liner is in direct contact with the inner surface of the diaper, the moisture will be transferred to and retained within the diaper. An optional third microfiber layer may be used as the outer layer to provide an additional degree of moisture retention and is preferably the same shape as the first two layers. Whichever layer is the outer layer (second or third) may have a relatively coarse finish on at least its outer surface to help keep the liner in position within the diaper or underpants. The two (or three) layers are stitched together along their outer curved perimeters. In a preferred embodiment, the inner, skin contact layer is treated with an anti-microbial finish to kill bacteria that may promote infection or irritation as well as for odor management.

In an alternative embodiment of the diaper liner application, the liner is formed from an elongated strip which may be generally rounded at the ends to form an oval shape which may be contoured and narrower at its center to fit smoothly within the crotch portion of the diaper or underpants. The composite textile has a skin contact layer formed from a woven fabric of synthetic filament yarns woven to provide air and moisture vapor permeability and a non-shearing surface, with a thickness of less than 1 mm, the skin contact layer having an inner, skin contact surface and a bottom surface and an outer layer formed from a breathable microfiber fabric woven from synthetic fibers having a cross-sectional shape that includes a plurality of voids for generating a capillary effect for moisture conduction from one surface to the opposite, outer portion of the fabric thickness, which is highly absorptive. Since the outer layer of the liner is in direct contact with the inner surface of the diaper, at least a portion of the moisture will be transferred to the diaper. The two layers are stitched together along their outer curved perimeters. In a preferred embodiment, the inner, skin contact layer is treated with an anti-microbial finish to kill bacteria that may promote infection or irritation as well as for odor management.

In yet another embodiment of the diaper liner application, a skin contact layer is formed from a woven fabric of synthetic filament yarns woven to provide air and moisture vapor permeability and a non-shearing surface, with a thickness of less than 1 mm, the skin contact layer having an inner, skin contact surface and a bottom surface. The skin contact layer is either permanently or removably attached to a highly absorbent fabric, which may be formed from a synthetic microfiber or a natural fiber, which absorbs all of the liquid that is wicked away from the wearer's skin by the skin contact layer. As above, the skin contact layer is preferably treated with an anti-microbial finish. In this application, the diaper liner itself has no absorptive component, but relies on the diaper fabric itself to provide the absorbing function.

In another embodiment of the diaper liner application, a first skin contact layer is a synthetic microfiber material that is highly absorbent and a second layer is formed from the same moisture-wicking fabric as is used for the first layer in the preceding embodiments, including an anti-microbial treatment. This embodiment is particularly useful for children who are undergoing “potty training” and other individuals who have a bed-wetting problem. The placement of the highly absorbent material against the skin provides an early alert to the wearer that a release of urine has occurred, with the idea that they will sense the wetness and get up to finish urinating in the toilet. The second, anti-microbial layer reduces odor, while the moisture wicking function helps keep the underwear or pajamas from getting wet.

In another application of the inventive liner, a bra pad has a cup-shape (concave) formed from a generally rounded shape of a multi-layer composite textile. The rounded shape may be circular, oval (elongated) or a polygon, such as a hexagon, octagon, decagon, etc., that is effectively rounded. The composite textile has a first, skin contact layer formed from a woven fabric of synthetic filament yarns woven to provide moisture-wicking, air and moisture vapor permeability and a non-shearing surface, with a thickness of less than 1 mm, the skin contact layer having an inner, skin contact surface and a bottom surface; interchangeable second and third layers are formed from moisture absorbing and retaining fabrics. The second layer is formed from a breathable microfiber fabric woven from synthetic fibers having a cross-sectional shape that includes a plurality of voids for generating a capillary effect for moisture absorption from one surface to moisture retention on the opposite surface of the fabric, thus ensuring that the bra and clothing stay dry. The third layer has the additional quality of moisture absorption retention and is preferably the same shape as the first two layers. In one embodiment, the third layer may have a semi-rounded shape, e.g., a half circle, providing additional absorbency in the area where the nipple contacts the lower portion of pad. The third layer may be positioned between the first layer and the second layer or it may be sandwiched between the second layer and the fourth layer. The fourth, outer layer is formed from a breathable, waterproof fabric having a thickness of less that 1 mm that is woven from synthetic yarn such as nylon or polyester to produce a moisture vapor transfer rate within the range of 5,000 to 20,000 grams per meter squared per 24 hours. In a preferred embodiment, the fourth layer is formed from a polyurethane (PU)-coated nylon fabric. A coating of TEFLON® (PTFE) may also be used. The four layers are stitched together along their outer curved perimeters. In one embodiment, the concave shape is formed by creating one or more pleats or darts in the upper portion of the pad to so that the pad generally fits the shape of the breast. The pleats may be formed at the same time the layers are stitched together by folding the fabric as the edges are stitched. Alternatively, the pleats may be formed after the layers are sewn together. In a second, preferred embodiment, pleats are omitted to avoid the presence of seams or stitching in the center pad body and are replaced by a number of smooth puckers that are formed in the first of a two-step sewing process. The first sewing step involves a stitch through all layers near their edges, which causes a slight puckering of the combined layers near the stitches. This creates a gentle curve that will allow the pad to at least partially conform to the inner surface of the user's bra cup without requiring any stitching in the center of the pad that might irritate the wearer's breast.

A slightly modified version of the nursing bra pad may be used as a cover for a breast form prosthesis. The first and second layers of such a cover are the same as those used for the nursing bra pad, i.e., a skin contact layer that has moisture-wicking and anti-microbial properties in a non-shearing, non-elastic synthetic fabric. The second layer is an absorbent layer formed from microfiber material that is hydrophilic on the skin side and hydrophobic on the opposite side, to transport moisture away from the skin contact layer. A third layer is stitched to the first two layers to define a pocket to permit insertion and removal of the prosthetic. The third layer may be formed from a fabric similar to that used for the first layer, with moisture-wicking properties, but may be woven to have multi-directional stretchability, ideally with a 4-way stretch, so that the prosthetic is firmly held in place to prevent slippage. Since the prosthesis is not attached to the bra itself, the wearer is free to select any style of bra that she may wish to wear.

In one aspect of the invention, the inventive wearable-article liner includes a moisture-wicking layer comprising a woven fabric of synthetic filament yarns woven to a pore size of 4 to 10 microns and having a thickness of less than 1 mm, wherein the moisture-wicking layer is treated with an anti-microbial finish; an absorbent layer comprising a synthetic breathable fabric comprising an integrated combination of hydrophobic and hydrophilic yarns having a weight of 180-340 g/m² for drawing moisture away from the moisture-wicking layer and transporting the moisture to the outer surface of the absorbent layer; and a seam formed around each edge of the moisture-wicking layer and the absorbent layer to attach the layers together.

In another aspect of the invention, a liner for wearable articles comprises a fabric woven from a continuous fine-denier filament yarn of polyester or polyester blend to a pore size of 4 to 10 microns, a coefficient of friction of 0.5 or less, and having a thickness of less than 1 mm, wherein the fabric is treated with an anti-microbial finish; and means for attaching the fabric to one or more absorbent materials. In an exemplary embodiment, the liner, which is typically intended for direct skin contact, is formed from a thin (<1 mm), lightweight (−2.5 ounce) polyester that is breathable, non-shearing, anti-microbial, moisture wicking and quick drying. Examples of appropriate fabrics for this upper layer include the fabric described in U.S. Pat. No. 6,277,770 of Smith, which is incorporated herein by reference. The first layer is cut into the desired shape for the intended purpose.

A second layer is cut to match the shape of the first layer from a moisture absorbing and distributing breathable fabric that may an integrated two-layer structure consisting of an inner, moisture absorbing surface and an outer transporting surface which draws moisture from the moisture absorbing surface, then distributes the moisture across the outer surface for dispersion and more rapid drying. Such fabrics are commercially available from a number of textile suppliers. In one example, the fabric is a microfiber formed from a blend of polyamide (NYLON®) and polyester. The second layer is thin (<1 mm) and lightweight when additional layers are to be included. In applications for diapers or diaper pads, the second layer may be on the order of 1 mm to 2 nun and may be textured to prevent slippage within the diaper or underpants.

In articles that are formed from more than two layers, the third layer of the assembly, which may be either the same shape as the first two layers or may alternatively be formed as a part of the shape with its edges matching one or more sides of the first layer, is an absorbent, breathable microfiber layer that is flexible and conforms to the body, with a thickness on the order of 1.0 to 4 mm, more preferably less than 2 mm and most preferably less than 1.5 mm. A preferred microfiber material has a density of 180-340 gm/m², which is commercially available from a number of sources. Such microfiber fabrics absorb liquid faster than cotton by virtue of the capillary effect created within tiny spaces between the threads and are able to dry rapidly by communicating the liquid away from the source. The positioning of the third layer provides optimal absorption in the area of the pad that contacts the skin. The second and third layers may be switched, so that the third layer is sandwiched between the first, skin contact layer and the second layer, or the third layer is sandwiched between the second moisture absorbing, breathable layer and the fourth, outer layer.

The outer layer of the assembly is a thin, lightweight, waterproof, breathable fabric such as the polyester or nylon fabrics with a coating such as polyurethane (PU) or polytetrafluoroethylene (PTFE) for enhancing the waterproof quality. Such fabrics are commercially available from a number of suppliers. In the preferred embodiment, a nylon/LYCRA® blend with a PU finish is used.

A factor that affects the smoothness and comfort of the surface is the assembly method used for combining the multiple layers and finishing the edges of a pad or cushion. The assembly method also effects launderability and drying time. In the preferred embodiment, the multi-layer fabric is stitched using a smooth polyester thread to minimize puckering or bunching, minimize washing and drying time, while maximizing comfort, durability and breathability. Seams, if any, should be free of puckering and are preferably limited to those used for finishing the outer edges of the pad, so that the skin contact surface is substantially seamless. In one embodiment, a friction-enhancing strip, such as silicone or other rubber-like synthetic material, may be formed or attached to selected areas on the outer edges of the pad to minimize shifting within the garment.

### BRIEF DESCRIPTION OF THE DRAWINGS

FIG. 1 is an exploded perspective view of the four layers of a nursing bra pad according to the present invention.

FIG. 2 is a view of the first inner layer of a nursing pad embodiment in which pleats are used to form the concave shape to indicate possible pleat locations.

FIG. 3a is a view of the first inner layer of an alternative embodiment formed using a two-step stitching process; FIG. 3b is a side view of the same embodiment.

FIG. 4 is a block diagram showing the steps for assembling the nursing bra pad of the embodiment of FIG. 3.

FIG. 5 is a top view of an embodiment of the outer layer that provides for insertion or removal of additional absorbent material.

FIG. 6 is a top view of an exemplary diaper liner with one end folded back.

FIG. 7 is a block diagram showing the steps for assembling embodiments of a liner according to the present invention.

### DETAILED DESCRIPTION OF PREFERRED EMBODIMENTS

According to the present invention, a liner for use with garments and other objects that come into contact with the skin is provided which is extremely lightweight, has minimal shear for added comfort against the skin, and is easy to launder without degrading comfort or performance. As used herein, “liner” includes a fabric or fabric assembly that may be separable from a garment or may be permanently incorporated into a garment, and includes covers and pads for items that may be placed against the skin for extended periods of time.

For purposes of the following description, the skin contact layer of a liner may be described as the “upper layer”, the “first layer”, or the “inner layer”, while the outer layer that is opposite from the skin contact layer is referred to as the “lower layer” or “outer layer”. These references are provided for convenience with reference to the orientation of the drawings and are not intended to be limiting or to indicate that the layering must always start with either the inner or outer layers during assembly.

In an exemplary embodiment, the inventive liner has a shape adapted to best fit the intended purpose. For example, for a bra insert, the shape may be circular or oval (elongated), or a many-sided polygon, such as a hexagon, octagon, decagon, etc., which is effectively rounded. For a diaper liner, an elongated strip is contoured to fit comfortably within the crotch area with sufficient area to cover the area that may become wet but without too much fabric so that there is bunching. For other liner applications, the fabric will be cut in appropriate shapes and dimensions for the desired use.

In most applications, the liner comprises a layered construction. The layers include an upper layer, in most applications intended for direct skin contact, which is a thin (<1 mm, preferably less than 0.5 mm), lightweight (−2.5 ounce) polyester that is breathable, non-shearing, moisture wicking and quick drying. Examples of appropriate fabrics for this upper layer include the fabric described in U.S. Pat. No. 6,277,770 of Smith, which is incorporated herein by reference. In the preferred embodiment the fabric is treated with an anti-microbial compound, as described below.

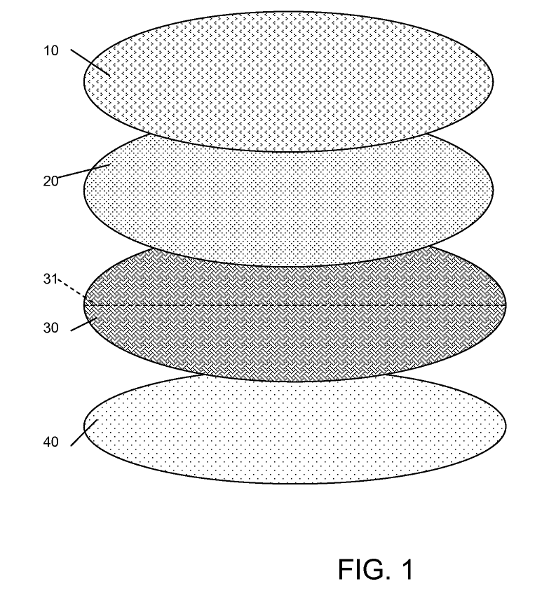

Describing a first exemplary application of the inventive liner for use as a nursing pad, as shown in FIG. 1, the thin, lightweight upper layer 10 should conform well to the user's body and dry quickly. Layer 10 is cut in a rounded shape with an upper portion and a lower portion corresponding to the nipple contact area. The rounded shape has a diameter in the range of 105-180 mm. If an oval or other elongated shape is used, typical dimensions will be around 120-180 mm along the long chord and around 105-120 mm for the short chord. The fabric used for the upper layer has the combined qualities of air porosity (to allow venting through the fabric's planar surface), moisture vapor transport (to enhance comfort), fabric flexibility (also a comfort-enhancing property), extremely small pore size, a non-shearing surface (for reduced friction), and durability to laundering. The fabric thickness is preferably less than 1 mm, with a typical thickness of around 0.2 mm. This combination of properties is provided by weaving fine-denier synthetic filament yarns (polyester or nylon-polyester blend) into a tight plain-weave construction with post finishing processes that maximize fiber coverage and filtration efficiency. Because the fabric is woven from continuous synthetic filament and/or spun yarns, there are no short fibers to irritate skin or become embedded in a wound or sore. The woven filaments are finished to provide a fabric with a mean pore size in the range of 4 to 10 microns, an air permeability of 0.5-30 cubic feet per minute per square foot of fabric at 0.5 inches of water (“cfm,” measured by Federal Test Method Standard (FTM) 5450, also known as ASTM D-737), a mean fabric flexibility in the range of 0.5 to 6.5 grams (bending resistance), and a moisture vapor permeability in excess of 800 g/m² per 24 hours. The coefficient of friction for the fabric is on the order of 0.5 or less, which is approximately one-half of the coefficient of friction of a cotton or polyester/cotton blend with roughly the same fiber and weave.

In the preferred embodiment, a suitable antimicrobial or fluorochemical finish is applied to or incorporated into the fabric of the upper, skin contact layer. Such finishes are known in the art. See, e.g., U.S. Pat. No. 4,822,667 of Goad, et al., U.S. Pat. No. 5,069,907 of Mixon, et al., or U.S. Pat. No. 6,762,172 of Elfersy, et al., among many others. An example of a suitable antimicrobial finish that can be applied is a compound of 3-(trimethoxysilyl)-propyl dimethyl octadecyl ammonium chloride (DOW CORNING 5700). Products made with such treatments are sold under the trademark AEGIS MICROBE SHIELD® by Aegis Environmental, Inc. (Midland, Mich.). In one embodiment, an antimicrobial treatment such as that described in U.S. Patent Publication No. 2003/0175438 may be used. The antimicrobial finish protects the fabric against bacteria and fungi, and inhibits the growth of odor-causing bacteria.

As shown in Table 1, compared with materials used in currently commercially-available nursing pads, the preferred skin contact layer (“non-shearing, moisture permeable synthetic”) as described above possesses all of the desired qualities for the user's comfort.

TABLE 1

| | Fabric | | |
|---|---|---|---|
| | Non-shearing, | | |
| | moisture-permeable | Disposable | |
| Feature | synthetic | (Paper) | 100% Cotton |
| Moisture | yes | yes | no |
| wicking | | | |
| Breathable | yes | yes | yes |
| Quick Drying | yes | NA | no |
| (<5 min.) | | | |
| Anti-microbial | yes | NA | no |
| Body | yes | no | no |
| conforming | | | |
| Non-shearing | yes | yes | no |
| Weight | light | light | heavy |
| Thickness | <1 mm | <1 mm | 2 mm |
| Washable | yes | no | yes |
| Recyclable | yes | no | NA |
| potential | | | |

The light, thin top layer is particularly advantageous if quilting or other stitching is used within the center area of the pad, since the thicker the fabric, the greater the chance of puckering and bunching when quilted, which can irritate the mother's nipples.

The second layer 20, also referred to as the first absorbent layer, is cut to match the shape (outline) of the first layer from a moisture absorbing and retaining breathable fabric that itself has an integrated two-layer structure consisting of an inner, moisture absorbing surface and an outer transporting surface which draws moisture from the moisture absorbing surface, then distributes the moisture across the outer surface for dispersion and more rapid drying without soaking through the outer layer. One process for forming such an engineered fabric involves a combination of hydrophobic and hydrophilic yarns, which may be effectively integrated by weaving, plate knitting or other techniques known within the textile industry. These moisture management fabrics act to prevent, or minimize, the collection of moisture against the body and in the fabric layer that is next to the wearer's skin. The moisture, in liquid or vapor form, leaves the skin surface and diffuses, or wicks, through the hydrophobic fibers to be absorbed by the hydrophilic fibers in the outer fabric layer. An example of such a fabric is described in U.S. Patent Publication No. 2003/0182922 of Peters, which is incorporated herein by reference. Fabrics with these characteristics are commercially available from a number of textile suppliers. The second layer should be thin (<1 mm) and lightweight.

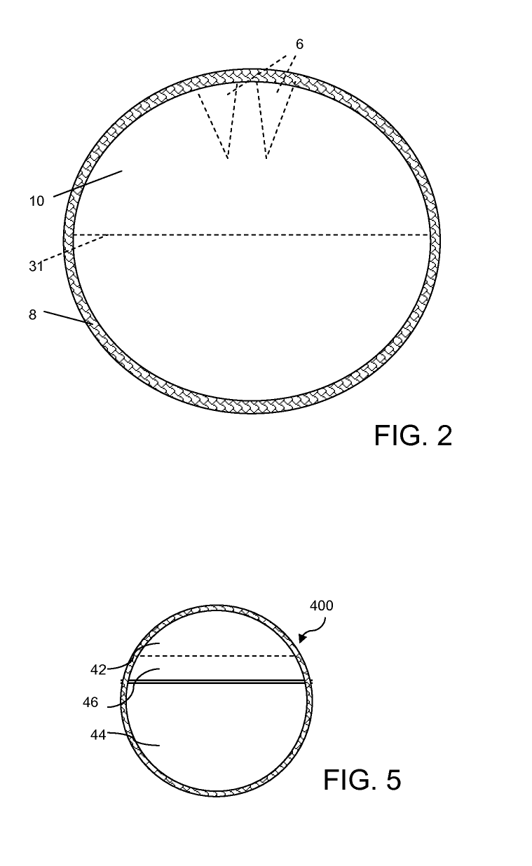

The third layer 30 of the assembly, which may also be referred to as the second absorbent layer, is used to enhance the moisture absorbency. This layer is an absorbent, breathable polyester microfiber that is flexible and conforms to the body, with a thickness on the order of 1.0 to 4 mm, more preferably less than 2 mm and most preferably less than 1.5 mm. The shape of the third layer should match that of the first and second layers, or alternatively, may be semi-rounded, e.g., a half-circle, as indicated by dashed line 31 in FIGS. 1 and 2, to provide extra absorbency at the lower portion of the pad corresponding to the nipple contact area. A preferred microfiber for the third layer has a density of 180-340 gm/m². Such microfiber materials are available from a number of commercial suppliers. One example of an appropriate microfiber material is described in U.S. Pat. No. 6,381,994 of Lee, which is incorporated herein by reference. Such microfiber fabrics are formed by weaving synthetic filaments or fibers that have many channels extending along the fiber lengths, thus creating fine voids within their cross-sections. Microfiber fabrics of this construction are able to absorb liquid faster and in greater volume than cotton due to the capillary effect produced by the voids and channels within the fibers and, thus, are able to dry rapidly by communicating the liquid away from the source.

Table 2 provides a comparison of relevant characteristics of the absorbent third layer of the multi-layer fabric of the preferred embodiment of the present invention to the materials used in currently commercially-available products.

TABLE 2

Fabric

| | Absorbent | Disposable | |
|---|---|---|---|
| Feature | microfiber | (paper) | polyester |
| Absorbs fluids | yes | yes | no |
| Washable | yes | no | yes |
| Body- | yes | no | no |
| conforming | | | |
| Breathable | yes | no | no |
| Discourages | yes | NA | NA |
| microbes | | | |
| Thickness | 2 mm | | 3-4 mm |
| Recyclable | yes | no | NA |
| potential | | | |

The third layer (second absorbent layer) may be omitted entirely in cases where the woman is experiencing only light milk leakage. Customization of the absorbency can be provided by including a closable opening in the outer layer to allow additional absorbent material to be inserted. As illustrated in FIG. 5, the modified outer layer 400 is formed from upper and lower semi-circles 42 and 44, respectively, which are sewn around their edges (during the stitching step) but left open across the body of the pad where they overlap to allow the flap 46 to be opened to insert or remove additional absorbent material such as the microfiber material used in the third layer. A similar modification in the skin contact layer can be made to permit insertion of a topical wound dressing for treatment of a breast or nipple infection. In this variation, the overlapping area would need to be shifted as much as possible toward the top of the pad so that the fabric edges would not come into contact with the already irritated nipple.

The outer layer of the assembly is a thin, lightweight, waterproof, breathable synthetic fabric such as the polyester or nylon coated with polyurethane (PU) or polytetrafluoroethylene (PTFE). Such fabrics are available from a number of different textile manufacturers worldwide.

TABLE 3

Fabric

| | Breathable, | | |
|---|---|---|---|
| | waterproof | | |
| Feature | synthetic | Disposable | polyester |
| Waterproof | yes | yes | yes |
| Breathable | yes | yes | no |
| Body- | yes | no | no |
| conforming | | | |
| Washable | yes | no | yes |
| Weight | light | light | heavy |
| Thickness | <1 mm | <1 mm | 2-3 mm |
| Recyclable | yes | no | NA |
| potential | | | |

Exemplary characteristics of a preferred fabric for the outer (bottom) layer of the inventive multi-layer support surface include a fabric weight on the order of 80-100 g/m² that is waterproof and breathable. The breathability of the outer layer is measured according to moisture vapor transfer rate (MVTR), expressed in units of grams/meter²/day. For purposes of the present invention, a MVTR range of 5,000 to 20,000 g/m²/day is preferred. Table 4 below provides a comparison of the breathability of three different fabrics after applying 2 ml of water to the fabric. The degree of dryness was tested on the underside of the fabric, which would correspond to the moisture to which a mattress beneath the fabric would be exposed. The MVTR values were provided by exemplary manufacturers and were not independently confirmed.

TABLE 4

| | Fabric (MVTR) | | |
|---|---|---|---|
| Hr:Min after | Fabric #1 | Fabric #2 | Fabric #3 |
| applying water | (7,000 g/m²/day) | (7,000 g/m²/day) | (10,000 g/m²/day) |
| 0:00 | 80% dry | 80% dry | 80% dry |
| 0:05 | 85% dry | 85% dry | 85% dry |
| 0:10 | 90% dry | 90% dry | 90% dry |
| 0:15 | 95% dry | 95% dry | 95% dry |
| 0:45 | 98% dry | 98% dry | 98% dry |
| 2:30 | Completely Dry | Completely Dry | Completely Dry |

The tests indicated that MVTR ratings of 7,000 and 10,000 g/m²/day were equally effective in drawing away the moisture at a desired rate.

In the preferred embodiment, the fourth, outer layer is a flexible, soft nylon66 (89%)/LYCRA® (11%) blend with a PU finish to enhance the waterproof characteristics of the fabric. Alternatively, a TEFLON® (PTFE) finish may be used. The fabric may have multi-directional stretchability, ideally with a 4-way stretch, e.g., SPANDEX®.

A factor that affects the smoothness and comfort of the surface is the assembly method used for combining the multiple layers and finishing the edges of a pad. The assembly method and materials also influence launderability and drying time. For example, a cotton thread tends to have a heavier weight, less give (stretchability) and a longer drying time. In the preferred embodiment, the multi-layer fabric is stitched using a smooth polyester thread. The polyester thread is stronger, lighter, and faster drying with better give compared to cotton or cotton-blends. All layers of the multi-layer assembly are simultaneously stitched around their rounded edges with polyester thread as illustrated in FIG. 2 to define seam 8. In one embodiment, at the same time that seam 8 is created, pleats may be formed along the upper portion of the pad by folding over the fabric along the dashed lines 6 to form a concave, cup-like shape to conform to the shape of the breast. It may be possible to create a quilted pattern within the central area of the pad, but stitching should preferably be limited to the upper portion, away from the nipple contact area in the lower portion, to avoid irritation by the thread edges.

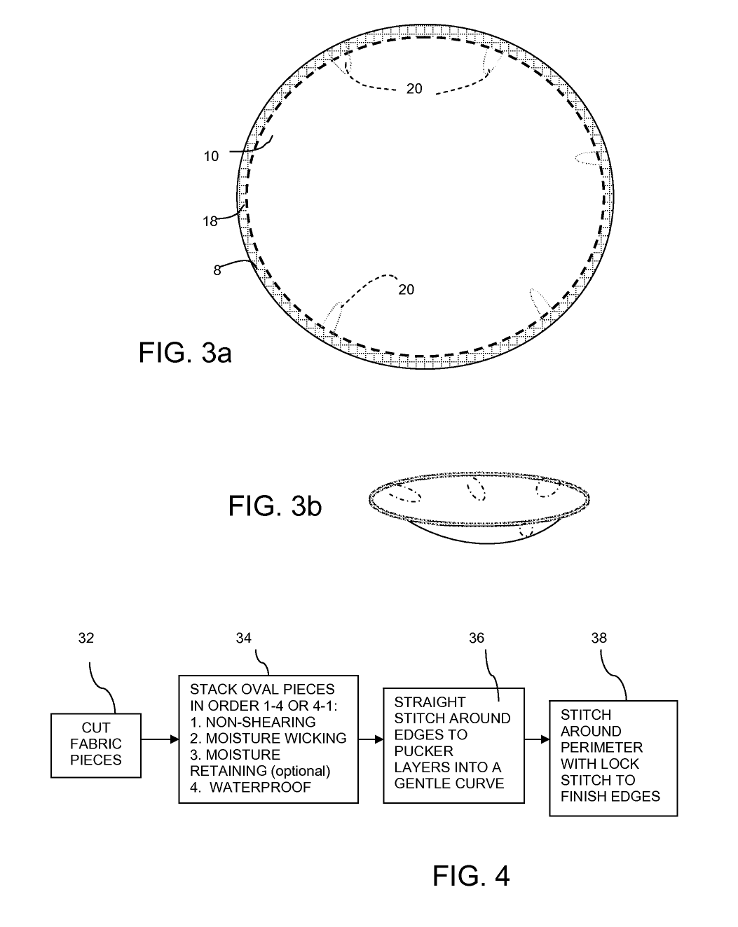

An embodiment of the multi-layer assembly is shown in FIGS. 3a and 3b. In this embodiment, the pleats or darts defined by folding over the fabric, as shown in FIG. 2, are replaced by a two-step stitching process. In the first step of the process, polyester thread is used to create a straight stitch 18 around the entire periphery of the rounded shape. The tension on the sewing machine is adjusted to produce a puckering around the edges of the assembly, drawing the edges of the layers in to create many tiny puckers 20. (The puckers shown in the figures are exaggerated in size for illustration and appear larger than would actually be created using the described technique.) The puckers are very small (less than 10 mm, preferably around 1-3 mm) and numerous, randomly distributed around the edges, thus conveying a gentle curvature to the assembly, as illustrated in FIG. 3b. Because the puckers 20 are small and generally confined to the outer periphery, there is little risk of creating an uneven surface at the center of the pad that might irritate the user's nipple. After the first stitching is completed, the edges of the pad are finished using a lock stitch 8.

FIG. 4 provides a block diagram of the steps of the process for assembling the nursing bra pad of FIGS. 3a and 3b. In step 32, rounded pieces are cut from each of the four fabrics described above, then stacked on top of each other from inner layer to outer layer or vice versa (step 34). In the first stitching step 36, a straight stitch is made around the edges of the layers to cause the assembly to pucker, forming a concave shape with a gentle curve. In the second stitching step 38, a lock stitch is used to secure the edges of the assembly.

In an alternative embodiment, the stitching may be replaced with an appropriate adhesive, thermal bonding, welding or other means known for attaching fabric layers, with caution to avoid altering the softness, pliability and other desirable characteristics of the fabric layers. Both the puckering and finishing steps should be achievable using alternative fastening techniques that are known to those in the art.

Generally, the combination of the cup-like shape and pleats or puckers should be sufficient to hold the pad within the bra cup to prevent shifting. However, in one embodiment, a friction-enhancing surface such as rubber or silicone may be formed on a portion of the outer layer to assist in holding the pad in position within the nursing bra. If used, the friction-enhancing surface should only cover a small area of the total pad area so that breathability is not impacted. Further, the surface would preferably be located only at the upper portion of the pad so that conformability and comfort are not affected in the areas nearest the nipple contact area.

The total combined thickness of the multi-layer assembly of the present invention is on the order of 2-5 mm, preferably at the lower end of the range. The minimal thickness enhances the assembly's pliability and its ability to conform comfortably to the user's breast without adding bulk. It also provides a pad that is easy to launder and dries quickly. The inventive pads provide excellent absorption of liquids while effectively drawing the moisture away from the skin to avoid skin irritation and bacterial or fungal growth. Further, the outer waterproof layer prevents leaking of milk that can soil clothing.

In an embodiment that may be used for a breast form prosthesis, the first and second layers of such a cover are the same as those described above for the nursing bra pad, i.e., a skin contact layer that includes moisture-wicking and anti-microbial properties in a non-shearing, non-elastic synthetic fabric. The second layer is an absorbent layer formed from microfiber material that is hydrophilic on the skin side and hydrophobic on the opposite side, to transport moisture away from the skin contact layer. A third layer is stitched to the first two layers to define a pocket to hold the prosthetic firmly in place. A similar configuration to that shown in FIG. 5 may be used, or a portion of the edge of the third layer may be left unstitched to leave an opening for insertion and removal of the breast form. The third layer may be formed from a fabric similar to that used for the first layer, with moisture-wicking properties, preferably woven to have multi-directional stretchability, ideally with a 4-way stretch.

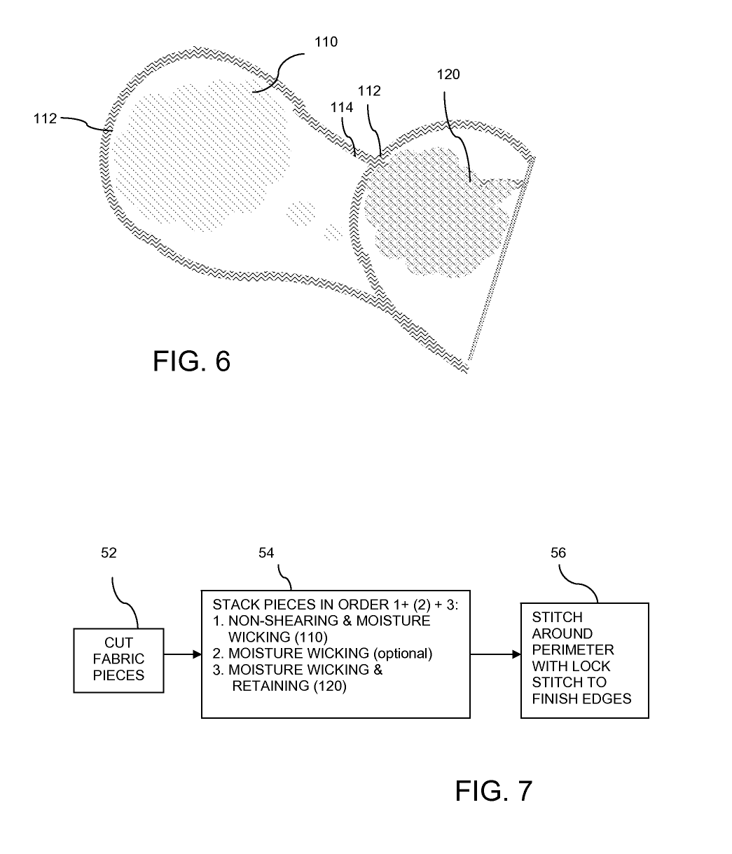

Referring to the diaper liner configuration of the inventive liner, an example of which is illustrated in FIG. 6, the thin, lightweight inner layer 110 should conform well to the user's body and should dry quickly. Layer 110 is cut in an elongated strip dimensioned to cover the majority, if not all, of the area that can become wet after urination. In the preferred embodiment, the ends 112 of the strip are rounded, while the center 114 is tapered inward at a location corresponding to the wearer's legs, thus defining a figure-eight-like shape that is relatively wide at its center. Exemplary dimensions are about 200 mm to 300 mm in length, about 7 mm to 10 mm in width at the widest points and about 6 mm to 9 mm at the center, however, the overall dimensions will be determined by the size of the garment to be lined, i.e., infant, child or adult. The fabric used for the upper layer has the combined qualities of air porosity (to allow venting through the fabric's planar surface), moisture vapor transport (to enhance comfort), fabric flexibility (also a comfort-enhancing property), extremely small pore size, a non-shearing surface (for reduced friction), and durability to laundering. The fabric thickness is preferably less than 1 mm, with a typical thickness of around 0.2 mm. This combination of properties is provided by weaving fine-denier synthetic filament yarns (polyester or nylon-polyester blend) into a tight plain-weave construction with post finishing processes that maximize fiber coverage and filtration efficiency. Because the fabric is woven from continuous synthetic filament and/or spun yarns, there are no short fibers to irritate skin or become embedded in a wound or sore. The woven filaments are finished to provide a fabric with a mean pore size in the range of 4 to 10 microns, an air permeability of 0.5-30 cubic feet per minute per square foot of fabric at 0.5 inches of water (“cfm,” measured by Federal Test Method Standard (FTM) 5450, also known as ASTM D-737), a mean fabric flexibility in the range of 0.5 to 6.5 grams (bending resistance), and a moisture vapor permeability in excess of 800 g/m² per 24 hours. The coefficient of friction for the fabric is on the order of 0.5 or less, which is approximately one-half of the coefficient of friction of a cotton or polyester/cotton blend with roughly the same fiber and weave.

An important feature of the skin contact layer is that inclusion of a suitable antimicrobial or fluorochemical finish, which is applied to or incorporated into the first fabric. Such finishes are well known in the art. See, e.g., U.S. Pat. No. 4,822,667 of Goad, et al., No. 5,069,907 of Mixon, et al., or No. 6,762,172 of Elfersy, et al., among many others. An example of a suitable antimicrobial finish that can be applied is a compound of 3-(trimethoxysilyl)-propyl dimethyl octadecyl ammonium chloride (DOW CORNING 5700). Products treated with this compound are sold under the trademark AEGIS MICROBE SHIELD® by Aegis Environmental, Inc. (Midland, Mich.). In general, antimicrobial agents that may be useful in this invention include, for example, quaternary ammonium silicon-containing materials and can be selected from the group consisting of quaternary ammonium silanes, quaternary ammonium containing oligomer siloxanes, quaterny ammonium containing polymeric siloxanes, quaternary ammonium di- or tri-silanes, silanes or siloxanes having hydrocarbon linkages such as —Si(C)_{y}Si—, wherein y has a value of 1 to 12, and quaternary ammonium containing siloxane/organic copolymers. In one embodiment, silanes including N,N-dimethyl-N-Octadecyl-3-(trimethoxysilyl) propanaminium chloride and N,N-Didecyl-N-Methyl-3-(trimethoxysilyl)propanaminium chloride, which are described in U.S. Patent Publication No. 2003/0175438, may be used. The antimicrobial finish protects the fabric against bacteria and fungi, and inhibits the growth of odor-causing bacteria.

The moisture wicking skin contact layer with the antimicrobial finish may be used alone as a diaper liner, either separable from or sewn directly to the diaper fabric, i.e., incorporated into the diaper as an integral component thereof. The liner may be shaped to be placed only in the crotch area of the garment, or the entire diaper or underpants can be lined. This antimicrobial liner provides a significant improvement to washable cloth diapers due to its ability to reduce skin irritation by drawing moisture away from skin to be absorbed into the diaper fabric, reducing odor-causing and other bacteria, and providing a smooth, non-shearing surface in direct contact with the skin. A liner of this type may also be used in combination with disposable paper and plastic diapers. If the liner is not integrally attached to the diaper, the liner may be removed for washing and reuse, even if the diapers themselves are not washable. In such an embodiment, the diaper alone may provide the moisture-absorbing function for receiving the moisture that is wicked away from the skin by the skin contact layer.

Referring still to FIG. 6, an outer layer 120 may be cut to match the shape (outline) of the upper layer from a moisture absorbing and retaining breathable fabric that itself has an integrated two-layer structure consisting of an inner, moisture absorbing surface and an outer transporting surface which draws moisture from the moisture absorbing surface, then distributes the moisture across the outer surface for dispersion to the diaper's absorbent portion. One process for forming such an engineered fabric involves a combination of hydrophobic and hydrophilic yarns, which may be effectively integrated by weaving, plate knitting or other techniques known within the textile industry. These moisture management fabrics act to prevent, or minimize, the collection of moisture against the body and in the fabric layer that is next to the wearer's skin. The moisture, in liquid or vapor form, leaves the skin surface and diffuses, or wicks, through the hydrophobic fibers to be absorbed by the hydrophilic fibers in the outer fabric layer. An example of such a fabric is described in U.S. Patent Publication No. 2003/0182922 of Peters, which is incorporated herein by reference. Fabrics with these characteristics are commercially available from a number of textile suppliers. In an exemplary embodiment, the second layer is formed from a blend of polyester and polyamide (NYLON®), with mixes on the order of 60:40 to 90:10, more preferably 70:30 to 80:20. The outer layer 120 may be on the order of 1 mm to 4 mm thick or less, preferably 3 mm or less, with a weight on the order of 180-350 g/m², capable of absorbing approximately four times its weight in moisture. In one embodiment, the second layer has a texture that is somewhat coarse, similar to fleece or another non-smooth finish, in order to hold the liner in place. Alternatively, as describe above, strips of silicone or other friction-producing materials may be applied to the outer surface to minimize movement.

An optional middle layer (not shown) may be added to provide additional moisture transfer from the skin contact layer 110 to the outer layer 120. This optional middle layer may have the same characteristics as the second layer of the nursing pad described above, i.e., a combination of hydrophobic and hydrophilic yarns to transport moisture from the inner surface to the outer surface to be absorbed by the outer layer. The optional middle layer may have a thickness on the order of less than 2 mm and preferably less than 1 mm.

FIG. 7 illustrates the steps for assembly of an exemplary diaper liner as shown in FIG. 6. In step 52, the fabric layers are cut into the desired shape to closely fit the dimensions of the crotch area of the garment with minimal bunching. In step 54, the layers are stacked in order to define a skin contact layer (110) and an outer layer (120). An optional a middle layer may be included for additional moisture transfer. In step 56, all layers are stitched together with a lock stitch to finish the edges.

In another embodiment of the diaper liner application, a first skin contact layer is a synthetic microfiber material that is highly absorbent and a second layer is formed from the same moisture-wicking fabric as is used for the first layer in the preceding embodiments, including an anti-microbial treatment. In other words, the two layer liner described with reference to FIG. 6 is formed, but it used upside-down, with the microfiber layer against the skin. This embodiment is particularly useful for children who are undergoing “potty training” and other individuals who have a bed-wetting problem. The placement of the highly absorbent material against the skin provides an early alert to the wearer that a release of urine has occurred, with the idea that they will sense the wetness and get up to finish urinating in the toilet. The anti-microbial layer reduces odor, while the moisture wicking function helps keep the underwear or pajamas from getting wet.

A factor that affects the smoothness and comfort of the surface of the liner is the assembly method used for combining the multiple layers and finishing the edges. The assembly method and materials also influence launderability and drying time. For example, a cotton thread tends to have a heavier weight, less give (stretchability) and a longer drying time. In the preferred embodiment, the multi-layer fabric is stitched using a smooth polyester thread. The polyester thread is stronger, lighter, and faster drying with better give compared to cotton or cotton-blends. All layers of the multi-layer assembly are simultaneously stitched around their rounded edges with polyester thread as to define seam 108.

Pads and liners constructed from the inventive multi-layer fabric are not limited to the examples described above. For example, pads may be constructed in an appropriate shape and size for application to wounds or irritated skin, thus providing the same advantages as those desired for nursing pads and diaper liners, namely, anti-microbial, moisture-wicking, and breathable. Where appropriate to enhance comfort and reduce risk of irritation, the liners may be formed from fabric woven using known methods to impart stretch for improved fit. For example, sleeves for use with prosthetic limbs may be formed entirely or partially using the inventive liners. Liners may be created for wear beneath athletic gear, such as protective padding for football, soccer and hockey, to reduce skin irritation and abrasion. In one embodiment, the liners may include pockets for insertion of additional fabric, e.g., for enhanced absorption, or for additional padding. The liners may also be sewn directly into athletic clothing. For example, a liner of the anti-microbial, moisture-wicking and breathable fabric may be incorporated within hockey shorts, and may include pockets for addition of pads. Liners of the same fabric may be made for helmets, hats, caps, visors, i.e., virtually any garment or equipment that may benefit from the non-shearing, moisture-wicking, anti-microbial and breathable qualities of the inventive liners. The liners can be formed as enclosures for ice packs or other thermal treatment to keep the skin dry by transferring moisture away from the skin. Liners and covers for cushions and pads, for example, for wheelchairs, seats and bed pads where an immobile patient is sitting or lying for long periods of time could help reduce bedsores. Liners may be provided for bicycle seats and other athletic equipment that partially or fully contacts the user's skin.

Additional items may be formed using the multi-layered fabric with slight modifications or with additional layers. For example, a diaper may be formed using a combination of layers similar to those of the nursing pad, but with the absorbent layers much thicker, or with an additional absorbent layer.

Training pants for children who are being toilet-trained or have bed-wetting problems may include an extra inner layer contacting the skin. This inner layer is highly absorbent to collect the initial release of urine and give the wearer an instant sensation of wetness so that he or she will be aware of the need to get up and go to the bathroom to urinate. One or two moisture wicking layers will keep the clothes dry. Training pants constructed using the inventive multi-layer fabric will be thinner and more comfortable than training pants that are currently in use, and are washable, unlike the popular training pants.

While the preferred embodiments of the invention have been described, modifications can be made and other embodiments may be devised without departing from the spirit of the invention and the scope of the appended claims.

1. A liner comprising:
a moisture-wicking layer comprising a woven fabric of synthetic filament yarns woven to a pore size of 4 to 10 microns and having a thickness of less than 1 mm, wherein the moisture-wicking layer is treated with an anti-microbial finish;
an absorbent layer comprising a synthetic breathable fabric comprising an integrated combination of hydrophobic and hydrophilic yarns having a weight of 180-340 g/m² for drawing moisture away from the moisture-wicking layer and transporting the moisture to the outer surface of the absorbent layer; and
a seam formed around each edge of the moisture-wicking layer and the absorbent layer to attach the layers together.

2. The wearable-article liner fabric of claim 1, wherein the moisture-wicking layer and the absorbent layer have a shape adapted to fit within a crotch area of a diaper or underpants.

3. The liner of claim 1, wherein the anti-microbial finish is selected from the group consisting of quaternary ammonium silanes, quaternary ammonium containing oligomer siloxanes, quaterny ammonium containing polymeric siloxanes, quaternary ammonium di- or tri-silanes, silanes or siloxanes having hydrocarbon linkages, and quaternary ammonium containing siloxane/organic copolymers.

5. The liner of claim 1, wherein the moisture-wicking layer has a coefficient of friction of 0.5 or less.

6. The liner of claim 1, wherein the moisture-wicking layer has a thickness of around 0.2 mm.

7. The liner of claim 1, wherein the moisture-wicking layer is woven from a continuous fine-denier filament yarn of polyester or polyester blend.

8. The liner of claim 1, wherein the absorbent layer has a thickness on the order of 1 mm to 4 mm.

9. The liner of claim 1, wherein the absorbent layer has a coarse fleece-like finish to hold the liner in place.

10. The liner of claim 1, wherein each layer has a rounded shape adapted to fit within a bra cup.

11. The liner of claim 10, further comprising an outer layer comprising a breathable, waterproof synthetic fabric having a thickness of less than 1 mm.

12. The liner of claim 11, wherein the outer layer has a moisture vapor transfer rate of 5,000 to 20,000 g/m²/day.

13. The liner of claim 11, wherein the outer layer has a finish of polyurethane.

14. The liner of claim 10, further comprising stitched means for forming a concave shape with the combined layers.

15. The liner of claim 11, wherein the stitched means comprises a straight stitch that creates puckers near the edges of the layers.

16. The liner of claim 10, further comprising an outer layer attached to the moisture-wicking layer and the absorbent layer to define a pocket for insertion of a pad or prosthetic.

17. The liner of claim 16, wherein the outer layer comprises a moisture-wicking, stretchable fabric.

18. The liner of claim 1, further comprising a moisture transfer layer sandwiched between the moisture-wicking layer and the absorbent layer, the moisture transfer layer comprising a synthetic microfiber fabric formed from an integrated combination of hydrophobic and hydrophilic yarns to transport moisture from the inner surface to the outer surface, the moisture transfer layer having a thickness on the order of less than 2 mm.

19. A liner comprising:
a fabric woven from a continuous fine-denier filament yarn of polyester or polyester blend to a pore size of 4 to 10 microns, a coefficient of friction of 0.5 or less, and having a thickness of less than 1 mm, wherein the fabric is treated with an anti-microbial finish; and
means for attaching the fabric to one or more absorbent materials.

20. The liner of claim 19, wherein the fabric has a thickness of around 0.2 mm.

21. The liner of claim 19, wherein the wearable article is a diaper or underpants, and the fabric is shaped to fit within the crotch-portion of the diaper or underpants.

22. The liner of claim 19, wherein the wearable article is a bra and the fabric is shaped to fit within the bra cup.

23. The liner of claim 22, further comprising an outer layer attached to the fabric to define a pocket for receiving a removable pad or prosthetic.

24. The liner of claim 19, wherein the one or more absorbent materials comprises an absorbent layer comprising a synthetic breathable fabric formed from an integrated combination of hydrophobic and hydrophilic yarns having a weight of 180-340 g/m² and a thickness on the order of 1 mm to 4 mm.

25. The liner of claim 19, further comprising a moisture transfer layer sandwiched between the fabric and the absorbent layer, the moisture transfer layer comprising a synthetic microfiber fabric formed from an integrated combination of hydrophobic and hydrophilic yarns to transport moisture from the inner surface to the outer surface, the moisture transfer layer having a thickness on the order of less than 2 mM.

26. The liner of claim 19, wherein the anti-microbial finish is selected from the group consisting of quaternary ammonium silanes, quaternary ammonium containing oligomer siloxanes, quaterny ammonium containing polymeric siloxanes, quaternary ammonium di- or tri-silanes, silanes or siloxanes having hydrocarbon linkages, and quaternary ammonium containing siloxane/organic copolymers.
